(19) United States
(12) Patent Application Publication
(10) Pub. No.: US 2005/0112183 A1
(43) Pub. Date: May 26, 2005

(54) Compositions and methods for treating neuropathic sensory loss

(76) Inventor: Bradley S. Galer, West Chester, PA (US)

Correspondence Address: GUY DONATIELLO;ENDO PHARMACEUTICALS, 100 PAINTERS DRIVE, CHADDS FORD, PA, 19317, US

(21) Appl. No.: 10/722,737

(22) Filed: Nov. 25, 2003

(51) Int. Cl. A61K 9/70

(52) U.S. Cl. 424/449; 514/534

(57) ABSTRACT

The present invention relates to methods of reducing the effects of neuropathically induced negative sensory phenomena (NSP). NSP are manifested as the decreased ability to feel light touch, pain, proprioception, vibration, warmth/heat, and coolness/cold. The NSP are treated by application of an anesthetic. The anesthetic is preferably a benzoic acid-based anesthetic. Specifically, a patch containing about 5% lidocaine may be used. The anesthetic is transdermally administered to a patient suffering from NSP at or near the locus of the negative sensory phenomena.

### FIELD OF THE INVENTION

The present invention relates to compositions and methods for treating pain, more particularly, the present invention relates to decreasing the effects of neuropathically induced negative sensory phenomena (NSP).

### BACKGROUND OF THE RELATED ART

Patients with damaged or dysfunctional peripheral nerves, a condition commonly known as “neuropathy,” often develop symptoms of sensory deficits or loss (numbness), such as a decreased ability to feel light touch, pain, proprioception, vibration, warmth or heat, and cool or cold conditions in the area of the nerve damage. Patients may also describe “feelings of numbness” over the affected body region. Such symptoms are conveniently labeled “negative sensory phenomena” or “NSP.” NSP are distinguished from Positive Sensory Phenomena (PSP) that are indicated by increased sensitivity, dysesthesia (tingling, pins and needles, etc.), and pain. Some neuropathy patients may experience both NSP and PSP, while others experience only one or the other.

U.S. Pat. No. 5,976,547—Archer et al. discloses a flexible wrap of an analgesic and an antiphlogistic including extracts of arnica montana. The wrap is used for treating peripheral and central pain, including lower extremity paresthesias, numbness and hyperesthesia associated with diabetic peripheral neuropathy. In addition to the extract of arnica montana, the wrap may contain one or more of several therapeutic or pharmaceutical agents including, inter alia, lidocaine. Lidocaine is not used to treat numbness.

U.S. Pat. No. 6,337,423—Axt et al., discloses the use of local anesthetics including lidocaine for treating neuropathic pain.

U.S. Pat. No. 6,147,102—Borgman, discloses the use of clonidine-containing preparations in treating sympathetically maintained peripheral neuropathy.

Published U.S. Patent Application U.S.2002/0037926, published on Mar. 28, 2002, discloses the use of sodium channel blockers in combination with gabapentin or pregabalin for treating chronic pain or convulsions.

Thus, although local anesthetics for treating neuropathic pain and associated PSP (tingling, pins and needles, and pain) are known, none relate to the treatment of neuropathically induced NSP (numbness, decreased sensation). In particular, the treatment of PSP or increased sensitivity, along with the pain, by administering local anesthetics is known. None of the references, however, suggest that a pain reducing treatment would also be applicable for increasing sensation where it is diminished or reducing the sensation of numbness in the region(s) of neuropathy. Therefore, there exists a long-felt and unmet need for methods for treating negative sensory phenomena (NSP).

### SUMMARY OF THE INVENTION

Accordingly, it has now been found that NSP in a neuropathy patient are alleviated by the transdermal administration of a pharmaceutical compound that is preferably applied to the affected area.

The present invention therefore provides methods for treating a sensory loss due to neuropathic NSP by applying an NSP-relieving amount of a pharmaceutical compound to a patient suffering from neuropathic NSP at a location near the sensory loss. Preferably the pharmaceutical compound is at least one compound selected from benzoic acid-based anesthetics, specifically benzocaine, procaine, lidocaine, prilocalne, or pharmaceutically acceptable salts and derivatives thereof. In those embodiments where lidocaine is applied, it is most preferable to utilize a lidocaine patch including a carrier containing 5% lidocaine.

In certain embodiments, the methods of the present invention include treating sensory loss due to neuropathic NSP, by transdermal administration of a local anesthetic. This involves applying a composition comprising from about 2 to about 10% by weight of the anesthetic (such as lidocaine) in a form capable of transdermal transport. The lidocaine is absorbed transdermally to provide relief at the site of the neuropathy. Preferably, if a patch is used, the active ingredient is covered with a cover selected from the group consisting of polyvinyl chloride, polyvinylidene chloride, polyethylene, synthetic rubber, woven polyester fabric, and non-woven polyester fabric. In another preferred embodiment, sensory loss due to neuropathic NSP is treated by transdermal administration and more specifically by applying a patch comprising a physiologically acceptable adhesive including from about 2 to about 10% by weight, and more preferably about 5% by weight, of lidocaine in a formulation that provides transdermal transport of the lidocaine, and a non-woven polyester covering. The medicated patch is applied directly to the skin where the patient describes the NSP. The medicated patch does not produce clinically meaningful blood plasma levels of active ingredient.

### DESCRIPTION OF THE PREFERRED EMBODIMENTS

The present invention relates to methods of reducing the effects of neuropathically induced negative sensory phenomena (NSP) such as numbness and decreased sensation. As described above, NSP is manifested as the decreased ability to feel light touch, pain, proprioception, vibration, warmth/heat, and coolness/cold. As described above, NSP may be manifest solely by patient complaint or description of “numbness” in the affected region without the ability to document abnormalities in nerves with electromyography, nerve conduction velocity, or quantitative sensory testing laboratory assessments. Therefore, as used herein, the terms “NSP” or “neuropathic NSP” should be interpreted broadly to include all such neuropathic conditions and indications whether now known or later discovered. Such NSP are, by definition, functional disturbances considered to be caused by neuropathy, unless a temporary external agent is acting, such as an injected temporary anesthetic.

In the present invention, a local anesthetic is applied to alleviate neuropathic sensory loss. The anesthetic improves sensation at the site of the application. The anesthetic alleviates the complaint of NSP as described by the patient as numbness. The anesthetic is a benzoic acid derivative, normally used as a local anesthetic, as distinguished from a general anesthetic. Specifically, benzoic acids such as benzocaine and cocaine, meta-aminobenzoic acids such as proparacaine, para-aminobenzoic acids such as procaine, chloroprocaine and tetracaine, and amide-derivatives of benzoic acid such as lidocaine, mepivacaine, bupivacaine and etidocaine are useful in the present invention. Of these, para-aminobenzoic acid derivatives and other amide-derivatives are preferred. More preferred are amide-derivatives. Specifically, it is most preferred if the local anesthetic is lidocaine, and specifically, a patch containing about 5% lidocaine. Lidocaine is a synthetic amide, 2-(diethylamino)-N-(2,6-dimethylphenyl)-acetamide (C₁₄H₂₂N₂O) used chiefly in the form of its hydrochloride as a local anesthetic and antiarrhythmic agent. The anesthetic is preferably applied to a patient suffering from NSP at or near, the locus of, the reduced sensation. The locus of the reduced sensation is the spot on the skin of a patient where the reduced sensation is most noticeable, where it is most uncomfortable, or where an identified neuropathy exists. These places usually coincide, but if not, the anesthetic should be applied at one or more of these locations until relief from symptoms is realized. The anesthetic actually increases sensation and improves comfort in NSP patients.

Methods for treating sensory loss due to neuropathic NSP by applying an anesthetic to a patient suffering from neuropathy induced NSP at a location near the sensory loss, as disclosed herein, can utilize an active ingredient selected from benzocaine, procaine, tetracaine, chloroprocaine, propoxycaine, cocaine, proparacaine, mepivacaine, bupivacaine, phenocaine, dibucaine, etidocaine, lidocaine, prilocalne, or pharmaceutically acceptable salts thereof, or alternatively a derivative of one of these active ingredients such as procaine butyrate, procaine borate, etc. Both the anesthetics and derivatives can be used alone or in combination. Those of skill in the art will be able to determine the amounts and concentrations of these active ingredients without undue experimentation in order to create dosages that can be administered transdermally in a manner that is both safe and efficacious. For example, in those embodiments where lidocaine is applied, it is most preferable to utilize a carrier containing about 5% lidocaine, preferably in a patch.

In certain embodiments, the methods of the present invention include treating sensory loss due to neuropathic NSP by transdermal administration of a composition comprising from about 2 to about 10%, preferably from about 3 to about 7% by weight of lidocaine in a form capable of transdermal transport, so that the lidocaine is transported transdermally to provide for relief at the site of the NSP. Preferably, the active ingredient is covered with a material selected from the group consisting of polyvinyl chloride, polyvinylidene chloride, polyethylene, synthetic rubber, woven polyester fabric, and non-woven polyester fabric, to cover and protect the area.

Thus it will be understood that the present invention encompasses transdermal patches, which are familiar drug delivery mechanisms to those skilled in the art. As an example, a treatment for sensory loss due to neuropathic NSP using a patch involves administration by applying a physiologically acceptable adhesive including from about 2 to about 10% by weight, and more preferably about 5% by weight, of lidocaine. The lidocaine is contained in a formulation that provides transdermal transport of the lidocaine from the adhesive. The patch includes a non-woven polyester covering.

The present invention is also directed to a composition for treating sensory loss due to neuropathic NSP by transdermal administration of an NSP-relieving amount of a composition comprising a plaster or gel containing from about 2 to about 10% by weight of lidocaine. Preferably, the composition contains lidocaine in about 5% by weight, and is combined with a cover selected from the group consisting of polyvinyl chloride, polyvinylidene chloride, polyethylene, synthetic rubber, woven polyester fabric, and non-woven polyester fabric. In preferred embodiments, the formulation provides at least eight hours of relief from NSP.

Where the invention comprises a plaster for treating sensory loss, the plaster contains a physiologically acceptable adhesive, comprising from about 2 to about 10% by weight of lidocaine, most preferably about 5% by weight, and a non-woven polyester covering. Certain preferred embodiments also provide a gel comprising from about 2 to about 10% by weight of lidocaine, most preferably about 5%, wherein the formulation comprises about 70 to about 90% weight of an anhydrous vehicle, such as ethanol, isopropanol, propylene glycol, or glycerin, along with about 0.1 to about 5% weight of a physiologically acceptable gelling agent, about 2 to about 20% weight of a nonionic surfactant, and up to about 10% weight of physiologically acceptable excipients.

It has previously been assumed that positive sensory phenomena (“PSP”) is caused by an increase in spontaneous nerve discharge from damaged and dysfunctional peripheral nerves. Therefore, it was expected that transdermally administered sodium channel blockers, including local anesthetics such as lidocaine, could reduce the increased spontaneous discharges responsible for PSP and hence result in relief of pain and dysesthesia. Based on the current understanding of the underlying etiology of NSP it would be unexpected that an anesthetic would effectively mitigate the negative effects of NSP. It has now been found, however, that the application of local anesthetics such as lidocaine can effectively treat NSP. For example, some diabetic neuropathy patients reported both pain relief and improved NSP, that is, the patients experienced pain relief, improvement of sensory loss (decreased numbness), and improved tactile response (they could better feel objects touching their skin). Thus the anesthetic as applied in this embodiment decreased numbness. This is contrary to all prior teachings about anesthetics.

It is believed that the mechanism of action of the present invention is that NSP may be caused, at least in some patients, by increased spontaneous discharges in a special population of peripheral nerves whose function is to relay perceptive information of “numbness” and other NSP. Therefore, in a manner similar to treating PSP, transdermally administered anesthetics, in appropriate concentrations, reduce the ectopic discharges in these dysfunctional NSP-responsible peripheral nerves. Of course, the present invention is not intended to be limited by this theory.

Although certain embodiments of the present invention have been set forth herein with particularity, these embodiments and the descriptions thereof are provided for purposes of explaining the present invention and are not limiting. Upon review of the foregoing, it will be readily apparent to those of skill in the art that there are numerous adaptations, modifications, and variations of the compositions and methods of treatment disclosed herein that would utilize the present invention. For example, most of the specific preferred embodiments are directed to the use of lidocaine, however, as explained above, numerous other anesthetics can be transdermally administered to achieve the same results. Therefore, in order to ascertain the true scope of the present invention, reference should be made to the appended claims.

1. A method for treating neuropathic negative sensory phenomena comprising applying an anesthetic to a patient suffering from neuropathic negative sensory phenomena at or near the locus of the negative sensory phenomena.

2. The method of claim 1 wherein said anesthetic is a benzoic acid derivative.

3. The method of claim 2, wherein said benzoic acid derivative is selected from the group consisting of benzocaine, procaine, tetracaine, chloroprocaine, propoxycaine, cocaine, proparacaine, mepivacaine, bupivacaine, phenocaine, dibucaine, etidocaine, lidocaine, prilocalne, and pharmaceutically acceptable salts thereof.

4. The method of claim 3, wherein the benzoic acid derivative is lidocaine.

5. The method of claim 4, wherein the lidocaine is contained in a patch.

6. The method of claim 5, wherein said patch contains between about 2% and about 10% lidocaine.

7. The method of claim 6, wherein said patch contains about 5% lidocaine.

8. A method according to claim 1, wherein method further comprises applying a cover over said anesthetic, said cover formed from a material selected from the group consisting of polyvinyl chloride, polyvinylidene chloride, polyethylene, synthetic rubber, woven polyester fabric, and non-woven polyester fabric.

9. A method for treating neuropathic negative sensory phenomena by transdermal administration of an anesthetic, said method comprising: applying a non-woven polyester cloth including a physiologically acceptable adhesive, comprising from about 2 to 10% by weight of lidocaine, at or near the locus of the negative sensory phenomena.

10. A method according to claim 9, wherein said lidocaine is present in about 5% by weight.

11. A method for decreasing numbness of the skin of a patient comprising applying a local anesthetic to said skin at or near a site of said numbness.
